(19) United States
(12) Patent Application Publication
(10) Pub. No.: US 2010/0069794 A1
(43) Pub. Date: Mar. 18, 2010

(54) Guide Wire with Core and Distal Sheathing

(75) Inventor: Bernhard Uihlein, Dettingen (DE)

Correspondence Address: CROWELL & MORING LLP;INTELLECTUAL PROPERTY GROUP, P.O. BOX 14300, WASHINGTON, DC, 20044-4300, US

(73) Assignee: EPFLEX FEINWERKTECHNIK GMBH, Dettingen/Ems (DE)

(21) Appl. No.: 12/443,633

(22) PCT Filed: Sep. 17, 2007
(86) PCT No.: PCT/EP2007/008057

§ 371 Date: Oct. 28, 2009

(30) Foreign Application Priority Data
Sep. 28, 2006 (DE) 10 2006 047 675.1

(51) Int. Cl. A61M 25/09

(52) U.S. Cl. 600/585; 604/164.13

(57) ABSTRACT

A guide wire includes a core which extends from a proximal to a distal end of the guide wire. The core is designed as a single part or from multiple longitudinal sections adjoining one another, and includes a distal cladding which surrounds the core in a distal section and is made from a polyurethane material or another soft-flexible plastic material. The core, in a section adjoining the distal section which is surrounded by the soft-flexible distal cladding, is unclad or is surrounded by a connection cladding which is stiffer than the distal cladding and is in the form of a helical spring or in the form of a tube, made of polytetrafluoroethylene or another plastic material with a similar stiffness, which contacts and surrounds the core, with the tube having an enlargement at its distal end, by which it surrounds a diameter-reduced proximal end region of the distal cladding and terminates, substantially flush in terms of the external diameter, on an annular shoulder of the distal cladding. By way of example, it is used for medical catheter instruments.

### BACKGROUND AND SUMMARY OF THE INVENTION

The invention relates to a guide wire comprising a core which extends from a proximal to a distal end of the guide wire and which is designed as a single part or from multiple longitudinal sections adjoining one another, and comprising a distal cladding which surrounds the core in a distal section and is made from a polyurethane material or another soft-flexible, i.e. soft-bendable, plastic material.

Such guide wires are used in particular in medical instruments, and specifically in catheter instruments. Boston Scientific Corp. offers a guide wire for such applications under the trade name of Jagwire®, which has a 5 cm long soft-flexible distal end section with a hydrophilic coating and the remaining proximal shaft section is designed to be stiffer and is provided with a black and yellow helical pattern for improved endoscopic visualization.

It is well-known to design a distal end section of a guide wire to be more flexible than the remaining proximal shaft section by tapering the wire core in this distal end section, i.e. by decreasing the diameter of said wire core, for example by using one or more axially adjacent conical taperings, with the core for example being composed of a super-elastic Ni/Ti material. This tapered distal core section is often surrounded by a distal cladding in the form of a helical spring, at the distal end of which a, for example, hemispherical end cap may be attached and the proximal end of which helical spring is attached to the wire core, for example in a diameter-reduced region of the latter, or to a proximally adjoining, different core cladding. Alternatively, the helical spring cladding can also extend continuously to the proximal end of the guide wire. The published patent applications WO 88/04940 A1 and WO 03/072179 A1, as well as patents U.S. Pat. No. 4,456,017, U.S. Pat. No. 5,456,732, DE 101 38 953 B4 and EP 0 714 315 B1 are mentioned as representatives of this prior art with distal helical spring cladding of the core.

The invention is based on the technical problem of providing a guide wire of the type mentioned initially which can be produced with relatively little effort and has a desired flexural behavior, in particular a particularly soft-flexible distal section connected to a significantly stiffer proximal shaft section if required.

According to the invention, in a distal section of the guide wire, the core is surrounded by a distal cladding made from a polyurethane material or another soft-flexible plastic material. This makes it possible for the corresponding distal section of the guide wire to have a very low stiffness if required. To this end, the core itself can also be designed to be more flexible in this distal section than in the section of the core proximally adjacent thereto, for example by correspondingly weakening the material and/or a corresponding choice of material.

According to one aspect of the invention, the wire core remains unclad in a region proximally adjoining the distal cladding, i.e. the surface of the guide wire in this region is formed by the core itself or, if need be, by a coating applied thereto.

According to a further aspect of the invention, the distal cladding is adjoined proximally by a tube cladding made of polytetrafluoroethylene (PTFE) or another plastic material with a similar stiffness and hence with a significantly higher stiffness than the distal cladding. The tube surrounds the core on all sides, contacts the latter, and has an enlargement on its distal end by means of which it surrounds a diameter-reduced proximal end region of the distal cladding and terminates, substantially flush in terms of the external diameter, on an annular shoulder of the distal cladding. This design measure allows a good connection of the more flexible distal cladding and stiffer proximally adjoining cladding of the core as well as a smooth external transition between the two claddings without abrupt steps. This can advantageously be aided by locating this transition region in a section of the core in which the latter is tapered.

According to a further aspect of the invention, a helical spring serves as the connection cladding of the core and proximally adjoins the distal cladding. This variation can likewise be implemented with advantageous properties regarding the flexural behavior of the guide wire and the connection of the two claddings, as well as the smooth external transition between the latter. Additionally, very flexible adjustment of the stiffness profile in the transition between the region of the distal cladding and the region of the proximally adjoining cladding is possible by appropriately designing the helical spring.

In a development of the invention, in a distal region, the core is designed to have constant stiffness or a stiffness which decreases stepwise in the direction of the distal end; this contributes to designing the guide wire to be more flexible in the distal end section than in the remaining region. In particular, this can be implemented by a corresponding continuous, in particular conical, or stepwise reduction of the diameter of the core, for example by abrading the latter. In this case, this diameter-reduced distal region of the core does not have to correspond precisely to the axial extent of the distal cladding.

In a development of the invention, the distal cladding is designed as a solid cladding which contacts the core on all sides and embeds the latter, and which forms a blunt distal guide wire end at its distal end and/or the proximal end region of which terminates with a stepwise reduction in diameter or with a diameter which continuously decreases over a predeterminable axial length. The former measure makes an atraumatic, blunt distal end of the guide wire possible in a simple fashion, while the latter contributes to an optimum stiffness profile and a smooth external profile of the guide wire surface level with the proximal end region of the distal cladding.

In a development of the invention, the cladding which proximally adjoins the distal cladding extends up to the proximal end of the guide wire, or its proximal end terminates at a proximally adjoining unclad core region or at a proximally adjoining further cladding. In the first- mentioned case, the connection cladding forms the entire guide wire surface with the exception of the more flexible distal end region in which the distal cladding forms the guide wire surface.

In an advantageous refinement of the invention, the surface of the distal cladding is hydrophilic, for example as a result of applying a hydrophilic coating onto the distal cladding.

In a development of the invention, the proximal end region of the distal cladding and the distal end region of the connection cladding merge into one another with the external diameters substantially being flush at a punctiform transition point or within a transition region of a predeterminable length, i.e. the external diameter of the guide wire does not noticeably abruptly change in this region.

In an advantageous development of the invention, the cladding which proximally adjoins the distal cladding is formed by a helical spring and a distal end region of which is formed with a stiffness which continuously decreases in the direction of the distal end, for example. This makes it possible to set the stiffness profile of the guide wire in the transition between the soft-flexible distal end section and the stiffer adjoining shaft section in a desired manner; for example, it can be set to be continuously decreasing from the higher value in the shaft section to the lower value in the distal end section over a selectable axial length, with a steeper characteristic line over a shorter length or a shallower characteristic line over a greater length.

To this end, in a refinement of this measure, the distal end region of the helical spring is formed with a winding spacing which increases in the direction of the distal end and/or with a decreasing spring wire thickness. A wire thickness in distal end region which decreases in the direction of the distal end can be implemented in the helical spring, for example, by an abraded external region as effected by external conical abrasion. In a further refinement, the region of the helical spring which is abraded externally has an external diameter which decreases in the direction of the distal end or which remains constant with a correspondingly increasing winding diameter. The former is particularly simple from a production point of view; the latter makes it possible to keep the external diameter of the helical spring constant, even in this region of decreasing stiffness.

In a development of the invention, an enveloping section of the distal region of the helical spring surrounds a proximal end section of the distal cladding. This further contributes to a good connection between the distal cladding and the proximally adjoining, core-cladding helical spring and to a gradual transition of the stiffness property of the guide wire from the more flexible distal end section to the stiffer, proximally adjoining section with the helical spring.

In a development of the invention, the helical spring has an embedding section on the distal end with a smaller external diameter than the proximally adjoining helical spring section, with the embedding section of the helical spring section being surrounded by the distal cladding. This also contributes, in an advantageous manner, to a good connection between distal cladding and helical spring and to an optimization of the flexural behavior of the guide wire at the transition from the distal end section with lower stiffness to the proximally adjoining shaft section with higher stiffness. Here, reference is once again made to the fact that these measures and, to this end, the other abovementioned measures can additionally be accompanied by an appropriate design of the wire core, in particular a stepwise or continuous reduction of the diameter of the latter in its region surrounded by the distal cladding and/or already in the region which is axially level with the surrounding helical spring.

In a development of the invention, the helical spring acting as connection cladding is attached to the wire core at one or more axially spaced attachment sites. This reliably holds the helical spring in its intended position with respect to the core, for example in the case of shearing forces which act on the distal end region of the guide wire and can be transferred from the distal cladding onto the helical spring.

### BRIEF DESCRIPTION OF THE DRAWINGS

Advantageous embodiments of the invention are described below and illustrated in the drawings, in which:

FIG. 1 shows a longitudinal sectional view of a guide wire with a soft-flexible distal core cladding and a proximally adjoining PTFE core cladding,

FIG. 2 shows a longitudinal sectional view of a guide wire with a soft-flexible distal core cladding and an otherwise unclad wire core,

FIG. 3 shows a longitudinal sectional view of a guide wire with a soft-flexible distal cladding and a helical spring core cladding adjoining a proximal annular shoulder of the former,

FIG. 4a shows a longitudinal sectional view of a distal part of a guide wire with a soft-flexible distal cladding and an adjoining helical spring core cladding formed by a distally stretched helical spring,

FIG. 4b shows a longitudinal sectional view of the helical spring used for the guide wire of FIG. 4a,

FIG. 5a shows a partial longitudinal sectional view of a guide wire in accordance with FIG. 4a for a variation with a distally stretched and tapered helical spring,

FIG. 5b shows a longitudinal sectional view of the helical spring used in the guide wire of FIG. 5a,

FIG. 6a shows a partial longitudinal sectional view of a guide wire in accordance with FIG. 4a for a variation with a distally stretched, enlarged and abraded helical spring,

FIG. 6b shows a longitudinal sectional view of the helical spring used in the guide wire of FIG. 6a before its distal abrasion,

FIG. 6c shows a longitudinal sectional view of the helical spring of FIG. 6b after its distal abrasion,

FIG. 7a shows a partial longitudinal sectional view of a guide wire in accordance with FIG. 4a for a variation with a distally stretched and diameter-reduced helical spring,

FIG. 7b shows a longitudinal sectional view of the helical spring used in the guide wire of FIG. 7a,

FIG. 8a shows a longitudinal sectional view of a guide wire similar to that of FIG. 5a with the helical spring being attached on the wire core,

FIG. 8b shows a longitudinal sectional view of the helical spring used in the guide wire of FIG. 8a, variation with a proximal end section of the wire core remaining unclad, and

FIG. 9b shows a longitudinal sectional view of the helical spring used in the guide wire of FIG. 9a.

### DETAILED DESCRIPTION OF THE DRAWINGS

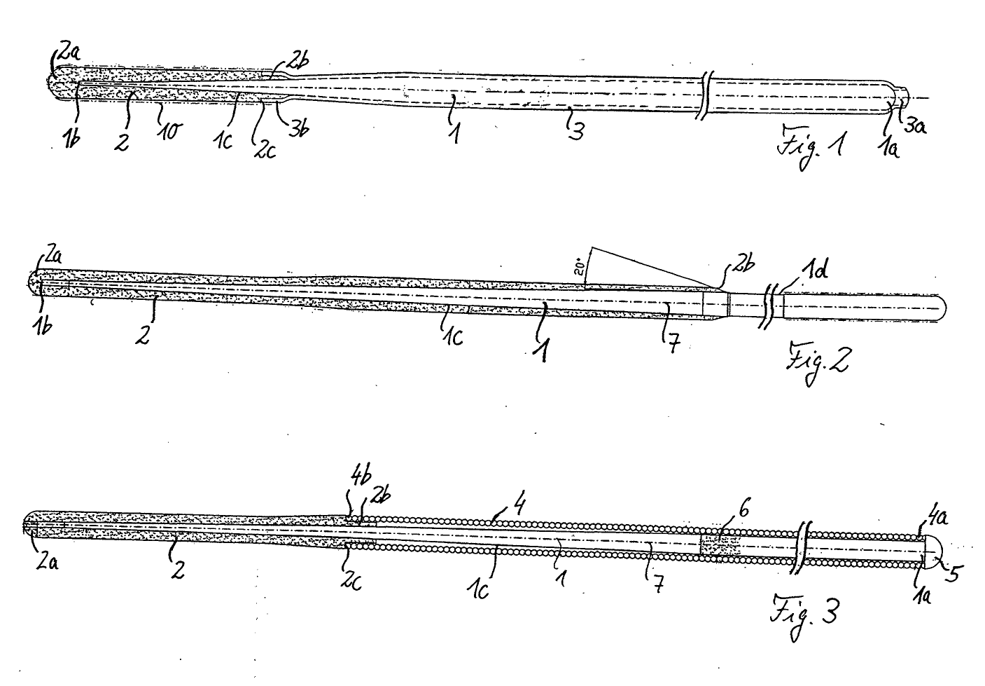

A guide wire shown in FIG. 1 has a wire core 1 which is made of super-elastic NiTi material, for example, and which extends from a proximal end 1a to a distal end 1b and is conically tapered by abrasion in a distal part up to the distal end 1b, i.e. its external diameter continuously decreases in this tapering region 1c from the larger value in the proximal shaft region to the smaller value at the distal end 1b. This distal tapering section 1c of the core 1 can, for example, have a length of a number of centimeters up to a length of a number of tens of centimeters, e.g. 20 cm.

In a distal end section with a predeterminable length of, for example, approximately 5 cm, the core 1 is surrounded by a distal cladding 2 of relatively soft-flexible plastic material which, in particular, can be a polyurethane material (PU material). Further soft-flexible plastic materials suitable for medical applications are, for example, nylon or the material known as Pebax. At its distal end 2a, the distal cladding forms a hemispherical, blunt and hence atraumatic distal termination of the guide wire.

A tube cladding 3 of the core 1 made of polytetrafluoroethylene (PTFE) proximally adjoins the soft-flexible distal cladding 2. This stiffer PTFE core cladding surrounds the core 1 up to and including its proximal end 1a whilst forming a rounded proximal end termination 3a. The PTFE core cladding 3 can, for example, be shrunk over the core 1 as a shrink tube and lies tight against the core 1, contacting it on all sides, even in a proximal part of its distal tapering region 1c, except for at an enlargement 3b of the PTFE cladding 3 at its distal end, as shown.

This distal end enlargement 3b of the PTFE cladding 3 is located in the tapering region 1c of the core 1 and butts, externally flush, against a corresponding annular shoulder 2c which is formed at the proximal end region 2b of the distal cladding 2. The material of the distal cladding 2 fills the intermediate space between the distal enlargement 3b of the PTFE cladding 3 and the core 1. This provides a good transition and a reliable connection between the soft-flexible distal cladding 2 and the proximally adjoining PTFE cladding 3, with it also being possible, as shown, to achieve a relatively smooth profile of the guide wire surface without abrupt steps, even in this transition region between the two claddings 2, 3.

It should be noted here that in this exemplary embodiment, and also in all other shown and described exemplary embodiments, the soft-flexible distal cladding 2 is preferably designed with a hydrophilic surface, for example by means of a hydrophilic coating 10 applied to the soft-flexible cladding material. The overall length of the wire core 1 and hence the guide wire can vary depending on the application and is typically between approximately 1 m and 5 m for medical applications, in particular in catheter instruments.

Hence, the exemplary embodiment of FIG. 1 overall shows an advantageous guide wire which maintains great flexibility in a distal section by tapering the wire core 1 and embedding it in a soft-flexible cladding and which guide wire has, in its remaining shaft section up to the proximal end, a core 1 cladding made of a PTFE tube material. This PTFE cladding and the increased core diameter mean that the guide wire is significantly stiffer in this section than in its soft-flexible distal end section, as desired. The transition between the soft-flexible distal cladding 2 of the core 1 and the proximally adjoining PTFE cladding 3 is designed in a structural and functional advantageous manner, in particular without the formation of cracks and without abrupt diameter changes of the guide wire.

In the following text, further advantageous variations of the guide wire are explained with reference to FIGS. 2 to 9b, with, for the sake of clarity, identical reference symbols being used for functionally equivalent components, even if the relevant elements are not identical.

Thus, FIG. 2 shows a variation of the guide wire with, again, a core 1 which is e.g. super-elastic and is conically tapered toward the distal end 1b in a distal end section and surrounded by a soft-flexible distal cladding 2 made, for example, of PU material. In this exemplary embodiment, the core 1 does not have any further cladding in the region 1d proximally adjoining the soft-flexible distal cladding 2. The distal cladding 2 terminates with a conically tapering proximal end 2b, as shown. This proximal termination 2b of the soft-flexible distal cladding 2 contributes to a smooth surface profile of the guide wire without the formation of abrupt external diameter changes; a sufficiently flat cone angle is selected to this end for the proximal termination 2b of the distal cladding 2, for example at approximately 20° to the guide wire longitudinal axis, as shown. Moreover, the properties and advantages mentioned above in the context of the exemplary embodiment of FIG. 1 analogously also apply to this exemplary embodiment.

FIG. 3 shows a first exemplary embodiment of a guide wire in which the cladding of the wire core 1 which proximally adjoins the soft-flexible distal cladding 2 is formed by a helical spring 4 which is also referred to as a helical spring wire or coil spring or coil spring wire. The distal end 4b of the helical spring 4 adjoins the proximal end 2b of the distal cladding 2 in an externally flush manner, with the cladding being designed like that of the exemplary embodiment of FIG. 1, i.e. provision is made for an annular shoulder 2c. The helical spring 4 extends in the proximal direction up to the proximal end 1a of the guide wire and terminates, externally flush, with its proximal end 4a at a hemispherical proximal termination cap 5 which is attached to the proximal wire core end 1a and to the proximal helical spring end 4a, for example by adhesive bonding.

The helical spring 4 is additionally attached to the core 1 at an attachment site 6 in a region between its ends, for example by adhesive bonding. This attachment/bonding site 6 is located, for example, in the proximal shaft section which is still in the region of, or just behind, the conical tapering 1c of the core 1. The additional attachment of the helical spring 4 to the core 1 can absorb possible impact/compressive loads which act on the distal end of the guide wire during its use and which can be transferred to the helical spring 4 from the distal cladding 2.

The use of the helical spring 4 as connection cladding of the core 1 which proximally adjoins the soft-flexible distal cladding 2 can provide functional advantages and also advantages relating the to production complexity compared to, for example, the alternative use of a PTFE core cladding. Moreover, particularly the properties and advantages of the soft-flexible distal cladding 2, mentioned above in context of the examples of FIGS. 1 and 2, apply correspondingly.

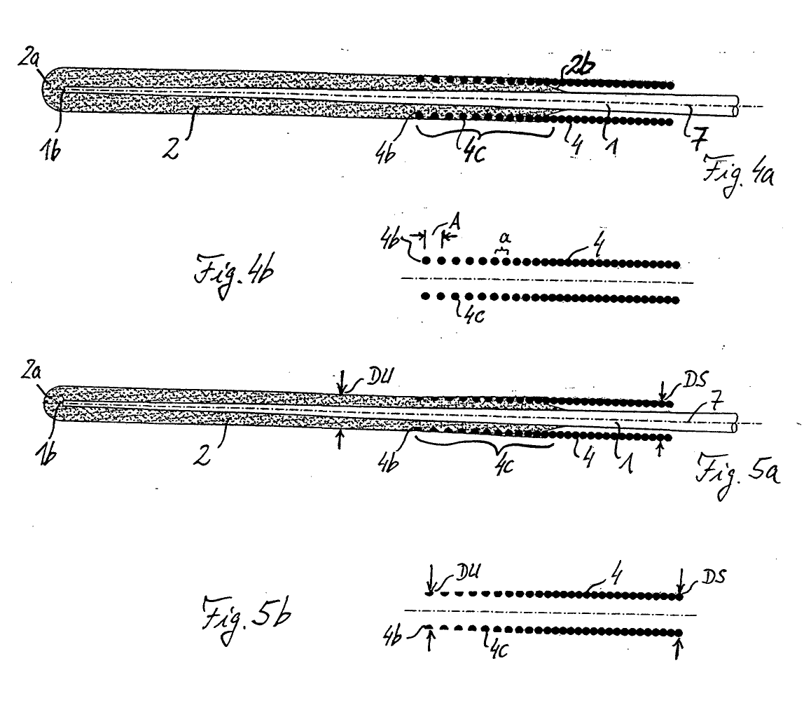

From a manufacturing point of view, a helical spring as connection cladding also makes it possible to very flexibly set respectively desired flexural characteristics of the guide wire in a relatively simple manner. This will be explained in more detail below on the basis of various exemplary embodiments, as illustrated in FIGS. 4a to 9b. What all these exemplary embodiments have in common is that, as a result of the special design of the helical spring which is used as connection cladding directly behind the soft-flexible distal cladding, the flexural characteristic of the guide wire is set in the sense of a smooth, gradual transition, i.e. as a result of the special design of the helical spring, the stiffness does not abruptly change from the low value in the more flexible distal end section to the higher value in the stiffer proximal shaft section, but rather it changes continuously over a corresponding transition region, the length of which can be selected as desired.

In this respect, FIGS. 4a and 4b show a first example of a guide wire. In this guide wire, a helical spring 4 proximally adjoins the soft-flexible distal cladding 2 and is stretched in a distal end region 4c, i.e. its winding spacing a of subsequent spring windings increases toward the distal end 4b, as shown more clearly in the individual view of FIG. 4b. In particular, the winding spacing a increases in the direction of the distal end 4b from the minimum value, which equals the diameter of the spring wire material in the case of a winding succession without gaps and which is found in the main part of the helical spring 4 in front of the distal end region 4c, to a maximum value A found at the distal end 4b. Both the maximum winding spacing A and the axial length of the stretched distal end section 4c can be selected freely according to the requirements of the respective application, e.g. a maximum winding spacing A approximately double the spring wire diameter and a length of the stretched distal end region 4c equaling a portion of the axial length of the soft-flexible distal cladding 2, e.g., approximately a quarter of this length, as shown. In the example shown, the winding spacing a increases substantially linearly along the distal spring end section 4c from the minimum value to the maximum value A, although it is understood that, in alternative exemplary embodiments, every other desired characteristic for the winding spacing as a function of the axial longitudinal coordinate can also be provided as required by correspondingly stretching the spring 4.

In this exemplary embodiment, as shown, the wire core 1 also tapers conically in a tapering region 1c which in this case extends over the region of the soft-flexible distal cladding 2 and, proximally, beyond the latter and which also extends over a distal part of the adjoining helical spring cladding 4. In line with the other exemplary embodiments, the conical tapering region 1c can, as required, comprise a single conical section or a number of successive conical regions which are directly adjacent to one other or are arranged at an axial distance from one another and have the same or different cone angles. It is furthermore understood that in this and in all other shown and described exemplary embodiments, the core 1 is formed as a single part which extends from its distal end 1b to its proximal end (not shown in FIG. 4a) or said core can be combined, by welding or adhesive bonding, from multiple longitudinal sections which adjoin one another, as known in the art and therefore requires no further explanations here.

FIG. 4a also shows that, in this example, the external diameter of the helical spring 4 is selected to be substantially equal to the external diameter of the soft-flexible distal cladding 2 so that a homogeneous transition with a constant external diameter of the guide wire is achieved between the two different, adjacent core claddings 2, 4. In this case, the soft-flexible material of the distal cladding 2 extends, in the proximal end region of the latter, into the intermediate space between the core 1 and the surrounding distal end region 4c of the helical screw 4 and, in the process, also fills the gaps between successive spring windings located there as a result of the stretching of the spring; as a result of this, there are no interfering gaps in the transition between the two different core claddings 2, 4. The proximal end 2b of the distal cladding 2 terminates, as in the example of FIG. 2b, with a termination cone running opposite to the conical tapering 1c of the core 1, with it being possible for the cone angle in this case also to be, for example, of the order of 20° to the longitudinal axis 7 of the guide wire.

As a result of the stretching, the stiffness of the helical spring 4 in the distal region 4c is correspondingly reduced continuously. As a result of this, a particularly homogeneous, gradual transition of the stiffness of the guide wire is achieved from a very soft-flexible distal end section, with the tapered core 1 and soft-flexible distal cladding 2, to the significantly stiffer proximally adjoining shaft section with the thicker core 1 and the helical spring core cladding 4 which is stiffer than the distal cladding 2. In other words, the stiffness of the guide wire does not abruptly change from the low value in the distal end region to the higher value in the proximally adjacent shaft section, but rather it changes gradually along the stretched distal spring end region 4c, with the axial length and its predeterminable flexural characteristics therefore substantially determining this stiffness transition of the guide wire.

FIGS. 5a and 5b illustrate a variation of the guide wire of FIGS. 4a and 4b, which differs only by a different design of the distal end region of the helical spring 4, with the same reference symbols being used here as elsewhere for corresponding or functionally equivalent components. In the example of FIGS. 5a and 5b, the proximal main part of the helical spring 4 is selected to have an external diameter DS which is slightly larger than the external diameter DU of the soft-flexible distal cladding 2 and the stretched distal end region 4c of the helical spring 4 is additionally conically abraded externally, as is shown in particular in the individual view of FIG. 5b; as a result of this the distal end 4b of the helical spring 4 substantially has the same external diameter as the distal cladding 2. FIG. 5a shows in particular that this results in a transition between the soft-flexible distal cladding 2 and the proximally adjoining helical spring cladding 4 of the core 1, which is once again externally flush, with the intermediate space between the distal spring end region 4c and the core 1 and between the successive windings being filled without gaps by the material of the distal cladding 2 in the transition region to the stretched distal spring end region 4c. Here, the external diameter increases evenly and with a smooth profile from the smaller value DU of the distal cladding 2 to the slightly larger value DS in the proximal main part of the helical spring cladding 4 of the core 1 in accordance with the externally abraded cone of the distal spring end region 4c.

As a result of the external conical abrasion, the stiffness of the distal end region 4c of the helical spring 4 decreases toward the distal end 4b in addition to the reduction in stiffness as a result of the stretching of the spring and so a particularly homogeneous or gradual stiffness transition of the guide wire is achieved overall from the low stiffness in the distal end section with the soft-flexible distal cladding to the higher value in the shaft section clad by the helical spring 4.

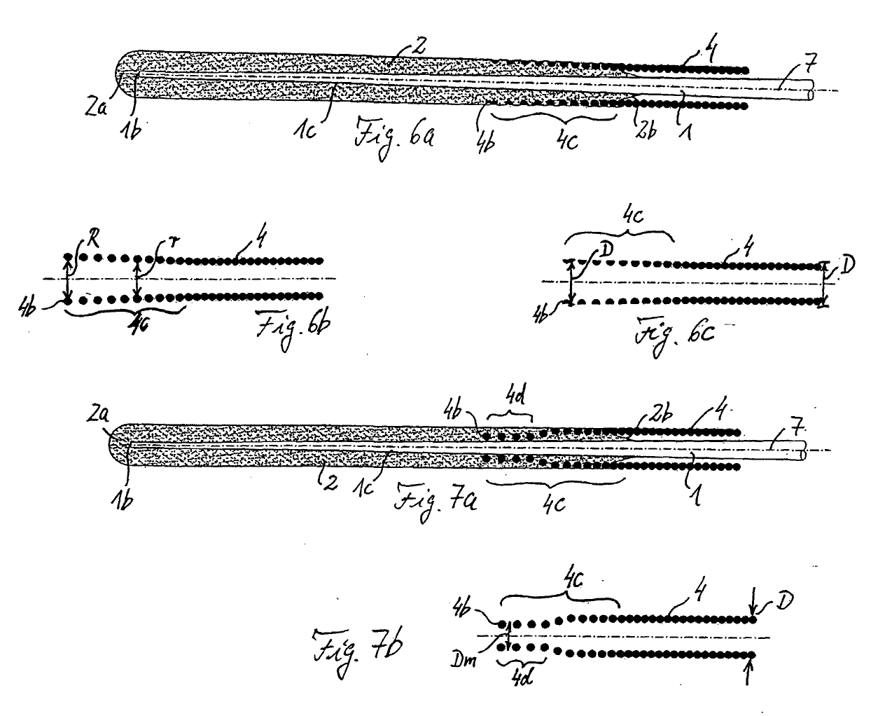

FIGS. 6a, 6b and 6c illustrate another variation of the guide wire, in which the property of a constant guide wire external diameter, even in the transition region between the distal cladding 2 and the proximally adjacent helical spring cladding 4, as implemented in the example of FIGS. 4a and 4b is combined with the measure of additionally reducing the stiffness of the distal spring end region 4c by external abrasion as implemented in the example of FIGS. 5a and 5b. Specifically, to this end, the distal end region 4c of the helical spring 4 used, as shown in the individual illustration of FIG. 6b, is radially enlarged in an unprocessed state before abrasion in addition to the axial stretching as specified in FIGS. 4a to 5b, i.e. the winding diameter r of the helical spring increases, preferably continuously, along the stretched distal end region 4c from a minimum value in the proximal main part of the helical spring 4 to a maximum value R at the distal end 4b. Subsequently, the helical spring 4, which is enlarged on the distal end in such a way, is externally abraded in its distal end region 4c to match the external diameter D present in the proximal main part. The helical spring 4 thus provided, and illustrated in FIG. 6c, therefore has a constant external diameter A everywhere, even in its distal end region 4c; however, its stiffness is continuously reduced toward the distal end 4b along the distal end region 4c—on the one hand, this is due to the axial stretching and, on the other hand, this is due to the increased material weakness effected by the external abrasion which is clearly shown in FIG. 6c.

The helical spring 4 prefabricated in this way is then pulled over the core 1 and the distal end region 4c of this helical spring 4 is connected to the distally adjoining soft-flexible cladding 2, as shown in FIG. 6a, with a gap-free transition region once again being formed. In this example, this transition region substantially has the same flexural characteristic as the example of FIGS. 5a and 5b combined with the feature of an external diameter of the guide wire remaining constant over the axial length as in the example of FIGS. 4a and 4b. The remaining properties and advantages of the guide wire embodiments in accordance with FIGS. 4a to 5b specified above correspondingly also apply to the example of FIGS. 6a to 6c and reference can be made thereto.

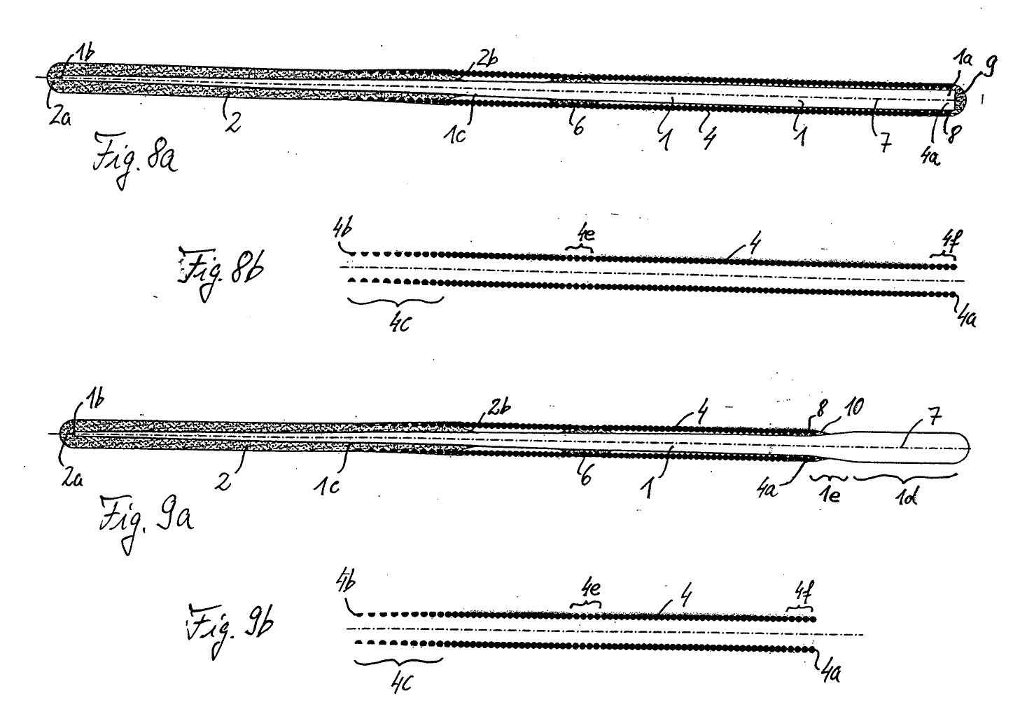

FIGS. 7a and 7b illustrate a variation of the guide wire which corresponds to the guide wire of FIGS. 4a and 4b, with the exception that the external diameter Dm of the helical spring 4 is reduced in a distal end part 4d of its axially stretched distal end region 4c compared to the external diameter D in the proximal main part and in the proximal part of the distal end region 4c, with this diameter reduction being implemented within a few spring windings in the illustrated example. The diameter-reduced distal end part 4d serves as an embedding section, by means of which the helical spring 4 is embedded in the proximal end region of the distal cladding 2 and, in the process, is surrounded by the latter in this region, as shown in FIG. 7a. The soft-flexible plastic material of the distal cladding 2 in turn fills out the intermediate space between the stretched helical spring section and the core 1, which is threaded through the interior, without gaps in the remaining transition region, as is the case in the examples of FIGS. 4a to 6c. The diameter-reduced distal end part 4d of the helical spring 4 can optionally butt against the core 1 with contacting.

The transition region designed in this way clearly results in a particularly secure connection of soft-flexible distal cladding 2 and proximally adjoining helical spring cladding 4 of the central wire core 1 with a smooth, constant external diameter and a gradual stiffness transition from the low value in the distal section in front of the helical spring 4 to the higher value in the proximal shaft section with the helical spring 4.

FIGS. 8a and 8b illustrate a variation of the guide wire according to the type of FIGS. 5a and 5b, with the guide wire being illustrated shortened in the proximal shaft section, but otherwise over its total length. In addition to the measures and properties of the distal part of this guide wire as explained above in the context of FIGS. 5a and 5b, the helical spring 4 in this guide wire cladding the proximal main part of the wire core 1 extends to the proximal end 1a of the core 1 and is attached directly to the core 1 at an attachment site 6, e.g. adhesively bonded, as explained above in the context of the exemplary embodiment of FIG. 3.

The attachment/bonding site 6 is preferably still located in the rear part of the distal tapering section 1c of the core 1 or it is alternatively located in the shaft section with the constant wire core diameter lying behind said tapering section with preferably a small distance from the tapering region 1c. The rear, proximal end 4a of the helical spring 4 is attached to the proximal wire core end 1a at a further attachment/bonding site 8. The attachment/bonding means used here simultaneously form a hemispherical proximal termination cap 9 of this guide wire.

The individual illustration of FIG. 8b shows that, in particular, the helical spring 4 is slightly axially stretched in the region of the two attachment/bonding sites 6, 8. Since, as a result of this, the bonding/attachment means can slightly penetrate between the spring windings in the stretched regions 4e, 4f, a very secure attachment of the helical spring 4 on the core 1 can be achieved which, in particular, can withstand shearing forces well which act on the soft-flexible distal tip of the guide wire when the latter is used and which can axially transfer onto the helical spring 3 via the distal cladding 2.

FIGS. 9a and 9b illustrate a further exemplary embodiment of a guide wire which uses a helical spring 4 which is identical in its design to that of the exemplary embodiment of FIGS. 8a and 8b. The only difference to the exemplary embodiment of the FIGS. 8a and 8b lies in the fact that the helical spring 4 in this case does not extend up to the proximal end 1a of the core 1, but rather ends at a distance in front of it, with a proximal end section 1d of the core 1 remaining without cladding. Specifically, this proximal end section 1d forms a region of the core 1 with a maximum diameter, which is first of all adjoined distally by a relatively short conical tapering region 1e in which the proximal end of the helical spring 4 is located. To be more precise, the proximal end 4a of the helical spring 4 is attached to the core 1 in this proximal tapering region 1e and/or just in front of it by an associated attachment/bonding site 8, with the bonding/attachment means used forming a conical termination 10 which is in the opposite sense to the wire core cone 1e located there; thus a relatively smooth external diameter profile of the guide wire is achieved at the transition from the helical spring 4 to the unclad proximal end section 1d of the core 1. Hence, the guide wire in this example has three longitudinal sections with different surfaces, specifically the distal region with the soft-flexible cladding 2 which is preferably coated in a hydrophilic manner, the proximal part 1d with the unclad core 1, and the intermediate, central guide wire section in which the core 1 is clad by the helical spring 4. Moreover, the properties and advantages, in particular regarding the soft-flexible distal cladding 2 and the proximally adjoining core cladding by the helical spring 4, specified above for the exemplary embodiment of FIGS. 8a and 8b also hold for the guide wire of FIGS. 9a and 9b.

As the different exemplary embodiments shown and explained above make clear, the invention provides an advantageous guide wire which has a very soft-flexible distal end region with a thin wire core and a soft-flexible plastic coating, preferably coated in a hydrophilic manner, and has a shaft section proximally adjoining thereto in which the interior wire core is surrounded by a helical spring or a specifically designed plastic tube in the distal end region, or in which the interior wire core remains unclad. The use of a helical spring for the connection cladding can be implemented in a comparatively cost-effective manner and allows for the profile of the stiffness of the guide wire in the transition region between the soft-flexible distal cladding and the proximally adjoining region to be controlled particularly well, as described above in the context of the appropriate exemplary embodiments. The invention is particularly suitable for guide wires in medical catheter applications, but also for all other applications, in which there is a need for such guide wires.

1-13. (canceled)

14. A guide wire, comprising:
a core which extends from a proximal to a distal end of the guide wire and which is designed as a single part or from multiple longitudinal sections adjoining one another, and
a distal cladding which surrounds the core in a distal section and is made from a polyurethane material or another soft-flexible plastic material, wherein
a section of the core adjoining the distal section, which is surrounded by the soft-flexible distal cladding, is unclad or is surrounded by a connection cladding which is stiffer than the distal cladding and is in the form of a helical spring or in the form of a tube, made of polytetrafluoroethylene or another plastic material with a similar stiffness, which contacts and surrounds the core, with the tube having an enlargement at its distal end, by which it surrounds a diameter-reduced proximal end region of the distal cladding and terminates, flush in terms of the external diameter, on an annular shoulder of the distal cladding.

15. The guide wire as claimed in claim 14, wherein, in a distal region, the core is designed to have constant stiffness or a stiffness which decreases stepwise in the direction of the distal end of the core.

16. The guide wire as claimed in claim 14, further designed as a solid cladding which contacts the core and embeds the latter, and which forms a blunt distal guide wire end at its distal end and/or the proximal end region of which terminates with a stepwise reduction in diameter or with a diameter which continuously decreases over a predeterminable axial length.

17. The guide wire as claimed in claim 14, wherein the connection cladding extends up to the proximal end of the guide wire or its proximal end terminates at a proximally adjoining unclad core region or at a proximally adjoining further cladding.

18. The guide wire as claimed in claim 14, wherein the surface of the distal cladding is hydrophilic.

19. The guide wire as claimed in claim 14, wherein the distal end region of the connection cladding and the proximal end region of the distal cladding merge into one another with the external diameters substantially being flush at a punctiform transition point or within a transition region of a predeterminable length.

20. The guide wire as claimed in claim 14, wherein distal end region of the helical spring is formed with a stiffness which continuously decreases in the direction of the distal end.

21. The guide wire as claimed in claim 20, wherein the distal end region of the helical spring is formed with a winding spacing which increases in the direction of the distal end and/or with a decreasing spring wire thickness.

22. The guide wire as claimed in claim 21, wherein the spring wire thickness in the distal end region, which thickness decreases in the direction of the distal end, is formed by a region of the helical spring which is abraded externally.

23. The guide wire as claimed in claim 22, wherein the region of the helical spring which is abraded externally has an external diameter which decreases in the direction of the distal end or which remains constant with a correspondingly increasing winding diameter.

24. The guide wire as claimed in claim 14, wherein an enveloping section of the helical spring surrounds a proximal end section of the distal cladding.

25. The guide wire as claimed in claim 14, wherein the helical spring has an embedding section on the distal end with a smaller external diameter than the proximally adjoining helical spring section, with the embedding section being surrounded by the distal cladding.

26. The guide wire as claimed in claim 14, wherein the helical spring is attached to the core at one or more axially spaced attachment sites.

27. The guide wire according to claim 14, wherein the guide wire is a medical instrument guide wire.

28. A guide wire, comprising:
a core which extends from a proximal to a distal end of the guide wire;
a distal cladding which surrounds the core in a distal section and is made from a soft-flexible plastic material; and
a tubular connection cladding which is stiffer than the distal cladding; and
wherein a section of the core adjoining the distal section, which is surrounded by the distal cladding, is surrounded by the tubular connection cladding which contacts the core, the tubular connection cladding at its distal end region surrounding a proximal end region of the distal cladding and terminating, flush in terms of the external diameter, on the distal cladding.

29. The guide wire as claimed in claim 28, wherein the distal cladding is made from a polyurethane material and the tubular connection cladding is made from a polytetrafluoroethylene material.

30. The guide wire as claimed in claim 28, wherein the tubular
connection cladding comprises a helical spring.

31. The guide wire as claimed in claim 30, wherein the distal end region of the helical spring has a winding spacing which increases in the direction of the distal end.

32. The guide wire as claimed in claim 30, wherein the distal end region of the helical spring has a decreasing spring wire thickness.

33. The guide wire as claimed in claim 32, wherein the decreasing spring wire thickness is formed by externally abrading a region of the helical spring.
